(19) United States
(12) Patent Application Publication
(10) Pub. No.: US 2014/0338153 A1
(43) Pub. Date: Nov. 20, 2014

(54) RAIL VEHICLE WITH DISINFECTION DEVICE

(71) Applicant: SIEMENS AKTIENGESELLSCHAFT, MUENCHEN (DE)

(72) Inventors: Florian Dopatka, Alt Duvenstedt (DE); Guenther Schless, Wesel (DE)

(21) Appl. No.: 14/363,816

(22) PCT Filed: Oct. 10, 2012
(86) PCT No.: PCT/EP2012/070008

§ 371 Date: Jun. 9, 2014

(30) Foreign Application Priority Data
Dec. 9, 2011 (DE) 102011088097.6

(51) Int. Cl. A61L 2/26; B61D 49/00

(52) U.S. Cl. CPC A61L 2/26; B61D 49/00
USPC 16/412; 16/110.1

(57) ABSTRACT

A rail vehicle has at least one handle, in particular a securing or activation handle for passengers and/or train personnel. The handle has a disinfecting device or is connected to a disinfecting device which is suitable for disinfecting the handle before, after and/or during the activation of the handle.

The invention relates to a rail vehicle having the features according to the preamble of patent claim 1.

As is known, rail vehicles have handles, in particular grab handles or actuating handles, which passengers or train staff can hold on to or with which certain functions of the rail vehicle can be actuated.

In rail vehicles or railway trains, the toilet doors are usually equipped with handles in the form of door handles, with which the toilet doors can be opened or closed manually. Which each manual actuation of the door handle, that is to say each time the toilet door is opened or closed, there is direct contact between the hand of the user and the door handle.

Tests have shown that more than 70% of users of train toilets do not wash their hands after using the toilet, and therefore the door handles can become polluted with viruses, bacteria, germs, fungi or other microorganisms. The possibility alone of such a pollution or contamination provokes disgust among a large proportion of passengers of railway trains and prevents them in part from using the toilets or wet rooms.

The problem, however, of a contamination of rail vehicle handles by viruses, bacteria, germs, fungi or other microorganisms is not limited to toilet door handles, and of course a contamination may also occur at other handles within the rail vehicle.

The object of the invention is therefore to specify a rail vehicle with which the contamination risk of handles is reduced and therefore concerns or reservations of the passengers with regard to a use of grab handles or actuating handles can be reduced.

This object is achieved in accordance with the invention by a rail vehicle having the features according to patent claim 1. Advantageous embodiments of the rail vehicle according to the invention are specified in dependent claims.

Accordingly, in accordance with the invention, at least one handle has a disinfection device or is connected to a disinfection device, which is suitable for disinfecting the handle before, after and/or during an actuation of the handle.

A key advantage of the rail vehicle according to the invention can be considered the fact that, as a result of the disinfection device provided in accordance with the invention, the likelihood of a relevant contamination of the handle or handles by germs, viruses, bacteria, fungi or other microorganisms is reduced, and the hygiene within the rail vehicle is thus considerably increased on the whole.

A further key advantage of the rail vehicle according to the invention lies in the fact that the passengers will feel better during the journey than in conventional rail vehicles because the risk of a possible infection by touching rail vehicle handles will be considered to be smaller, and inhibitions with regard to a use of the rail vehicle handles will lessen.

Since a contamination with germs, viruses, bacteria, fungi or other microorganisms is increased in the region of wet rooms, and is therefore of particular significance there, it is considered to be advantageous if the handle is a grab handle or actuating handle that is fixed in or to a wet room of the rail vehicle and enables an individual to hold himself steady within the wet room or enables an actuation of a function of the wet room.

By way of example, the handle may be a doorknob or a door handle of a rail vehicle toilet or a handle for lifting a toilet lid.

In order to enable continuous disinfection, it is considered to be advantageous if the disinfection device has a disinfectant store, in which a disinfectant, in particular a disinfecting liquid, is stored. In this embodiment the size of the disinfectant store determines the period of an effective disinfection by the disinfection device and therefore the maintenance interval of the disinfection device.

The disinfectant store, for example, may comprise a sponge, an open-pore rubber element and/or a pressure bottle.

A germicidal liquid, for example an alcohol-containing liquid, is preferably used as disinfectant. Fragrances are preferably added to the disinfectant and release a fresh fragrance indicative of the disinfection.

In accordance with a first particularly preferred embodiment the disinfectant store is arranged in the handle interior and/or is covered externally by the handle outer surface. An integration of the disinfectant store and/or of the disinfection device totally within the handle to be disinfected enables a preassembly of the disinfection device in the handle, such that the handle pre-fitted with the disinfection device can be mounted within the rail vehicle with low outlay.

For the disinfection of the handle, it is considered to be advantageous if the handle, on the outer surface thereof at least in portions, has discharge openings, in particular pores, hole or nozzles, through which disinfectant can exit outwardly from the handle interior.

In accordance with a second particularly preferred embodiment of the rail vehicle the handle is fixed to a fastening wall, in particular to a door, and the disinfectant store is fixed on the same side of the fastening wall or door, within the fastening wall or door, or on the other side of the fastening wall or door. A positioning of the disinfectant store separately from the handle, depending on the spatial conditions, can simplify a refilling of the disinfectant store with disinfectant.

Additionally or alternatively, the disinfection device may have at least one spraying apparatus, in particular a spray nozzle, which is arranged separately from the handle on the fastening wall and sprays the handle with disinfectant after and/during actuation of the handle. A spraying or wetting of the handle using a spraying apparatus is possible with all types and forms of rail vehicle handles, such that even existing rail vehicle handles that themselves are not equipped with or cannot be equipped with an internal disinfection device can be subsequently retrofitted with a possibility for disinfection.

Alternatively or additionally, it is also possible to ensure a disinfection of the handle with the aid of a coating, which is applied completely or in portions to the outer face of the handle and is provided, in particular saturated, with a disinfectant.

In accordance with a particularly preferred embodiment, the disinfection device is connected to a plurality of handles and is suitable for disinfecting the associated handles before, after and/or during an actuation of the respective handle. By way of example, it is possible, with the aid of a single disinfection device and therefore by way of example with the aid of a single disinfectant container, to disinfect a door handle on the inner side of the door as well as the associated handle on the outer side of the door.

The invention additionally relates to the specific handles and disinfection devices described here, irrespectively of the advantageous use thereof in a rail vehicle. However, the handles and disinfection devices are preferably suitable for rail vehicles.

The invention will be explained in greater detail hereinafter on the basis of exemplary embodiments; here, by way of example

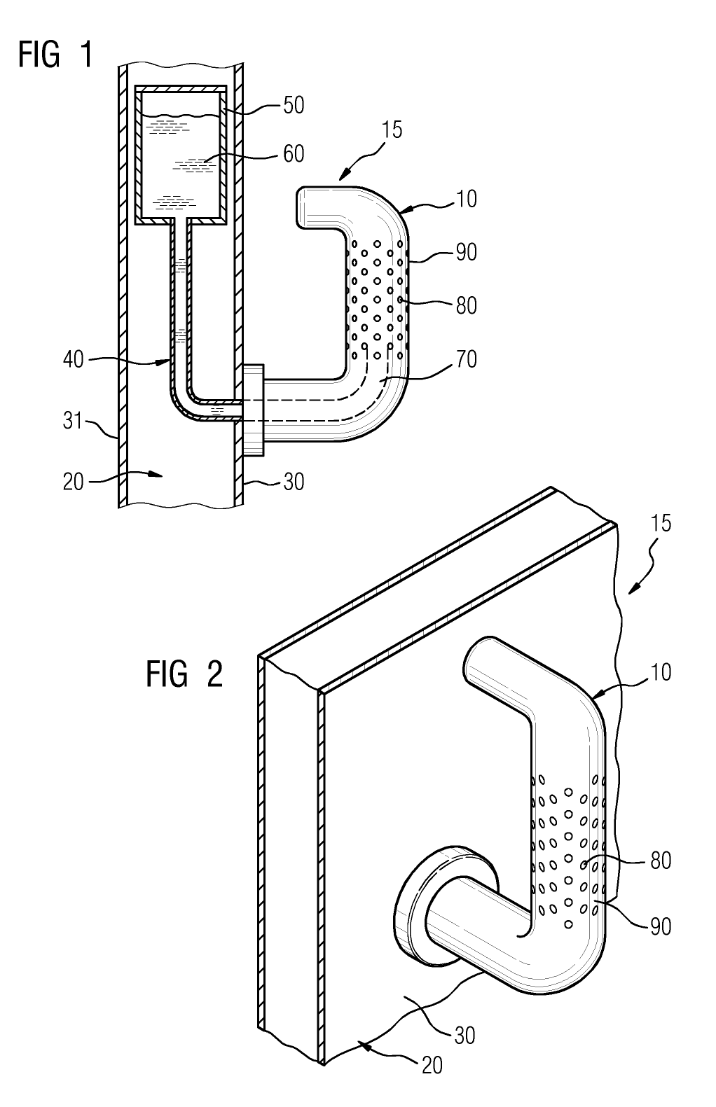

FIG. 1 shows a first exemplary embodiment for a handle equipped with a disinfection device,

FIG. 2 shows the handle according to FIG. 1 in a three-dimensional view from the side,

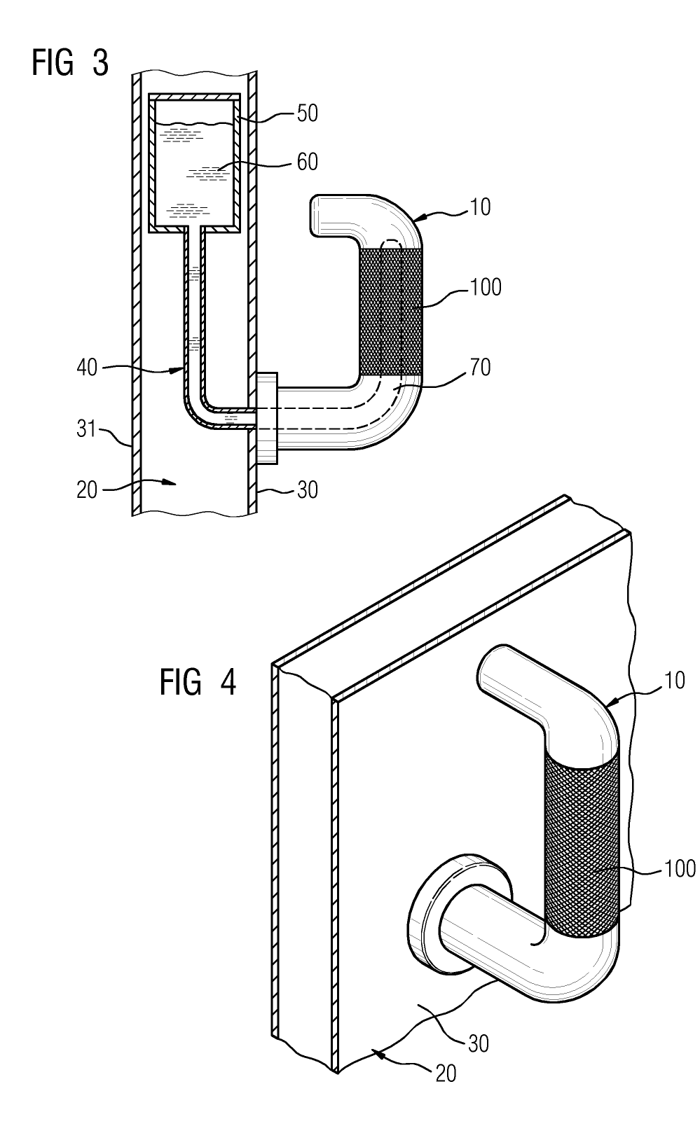

FIG. 3 shows a second exemplary embodiment for a handle equipped with a disinfection device,

FIG. 4 shows the handle according to FIG. 3 in a three-dimensional view from the side,

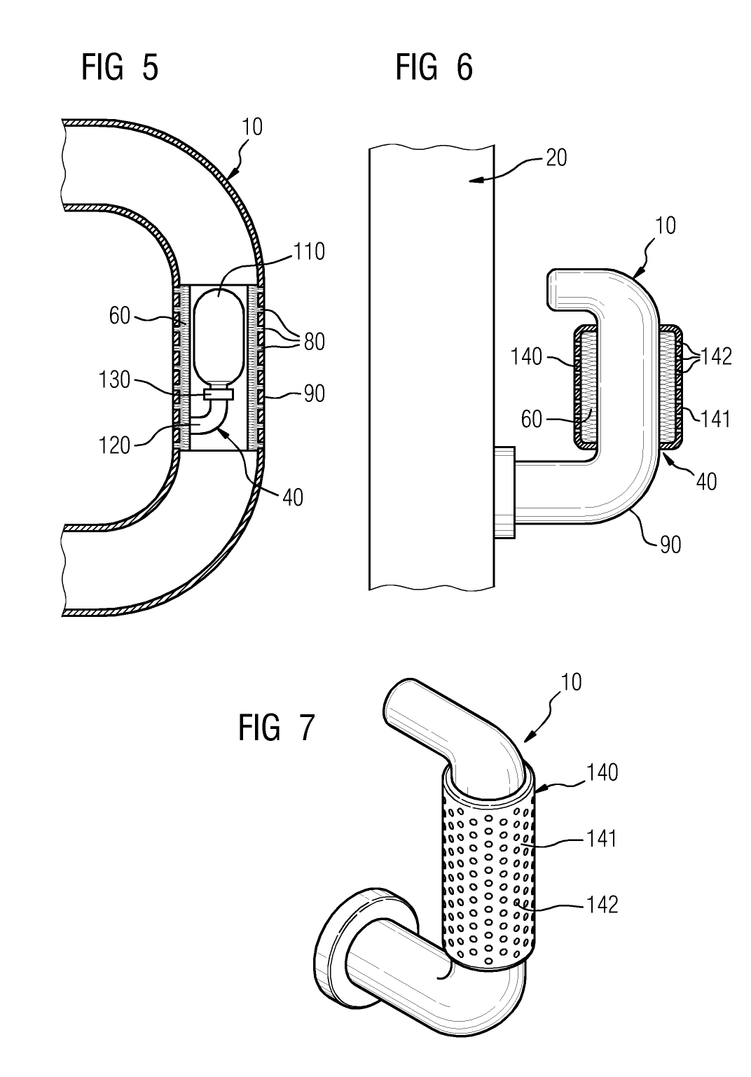

FIG. 5 shows a third exemplary embodiment for a handle equipped with a disinfection device,

FIG. 6 shows a fourth exemplary embodiment for a handle equipped with a disinfection device,

FIG. 7 shows the handle according to FIG. 6 in a three-dimensional view from the side,

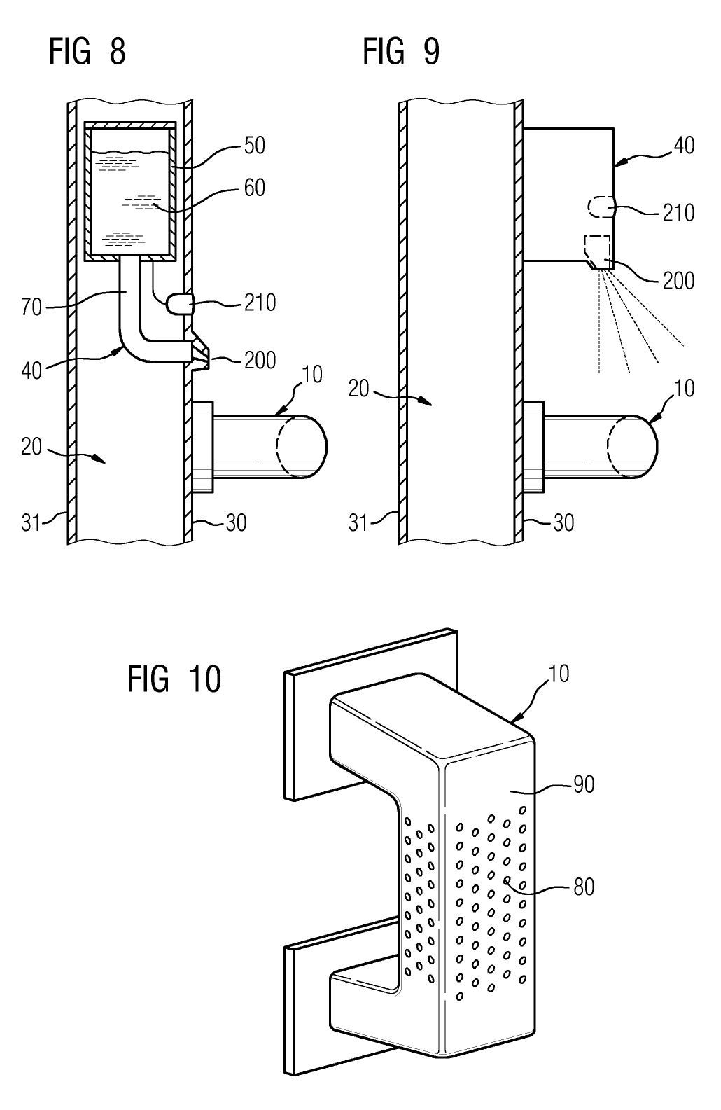

FIG. 8 shows a fifth exemplary embodiment for a handle equipped with a disinfection device, wherein the disinfection device is positioned separately from the handle,

FIG. 9 shows a sixth exemplary embodiment for a handle equipped with a disinfection device, wherein here too the disinfection device is positioned separately from the handle,

FIGS. 10-12 by way of example show different handle variants, which are or can be equipped with a disinfection device, and

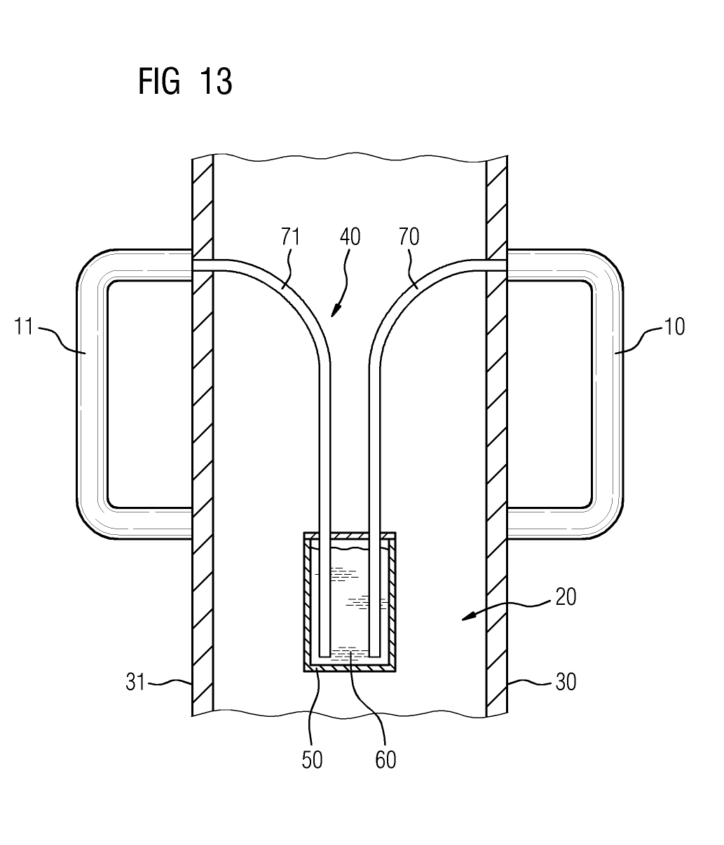

FIG. 13 shows an exemplary embodiment for a disinfection device that is connected to two handles and disinfects these two handles.

For sake of clarity, the same reference signs will always be used for identical or comparable components in the figures.

FIG. 1 shows an exemplary embodiment for a rail vehicle handle, referred to hereinafter as a handle 10 for short, of a rail vehicle 15 not shown in greater detail; the handle 10 forms a door handle for a rail vehicle door 20 and is mounted on the outer side 30 of the rail vehicle door 20. By way of example, the rail vehicle door 20 may be a toilet door of the rail vehicle 15.

FIG. 1 additionally shows a disinfection device 40, which comprises a disinfectant store in the form of a disinfectant container 50. A preferably liquid disinfectant 60 is located in the disinfectant container 50. The disinfectant 60 preferably contains chemicals, for example based on alcohol, which are suitable for killing or destroying bacteria, viruses, germs, fungi or other microorganisms in a lasting manner.

The disinfectant container 50 is mounted inside the rail vehicle door 20 and is connected via a line 70 to the handle 10. The line 70 extends from the disinfectant container 50, through the inside of the rail vehicle door 20, into the interior of the handle 10 and is connected to pores 80, which are arranged on the outer surface 90 of the handle 10 in order to wet the outer surfaces 90 of the handle 10 with the disinfectant 60.

In the exemplary embodiment according to FIG. 1, the disinfectant container 50 is arranged spatially above the handle 10, such that the disinfectant 60 can be transported into the handle 10 and therefore to the pores 80 merely as a result of the force of gravity. In addition, a pump may of course also be provided, which pumps the disinfectant 60 to the pores 80, however this is not necessary with the arrangement according to FIG. 1.

Alternatively, it is possible to position the disinfectant container 50 beneath the handle 10, however in this case a gravity-induced conveyance of the disinfectant 60 to the pores 80 would not be possible; in this case a transport of the disinfectant 60 would have to be brought about in a different way, whether via overpressure or a pump device, which is not shown in FIG. 1 for reasons of clarity.

In order to prevent the outer surface 90 of the handle 10 from being permanently moist, it is considered to be advantageous if the disinfectant 60 is volatile and evaporates relatively quickly.

The outer surface 90 can be wetted continuously by means of the disinfection device 40; alternatively, it is possible to only trigger a wetting of the outer surface 90 with disinfectant 60 before, during or after an actuation of the handle 10. In the latter case, the disinfection device 40 is preferably equipped with sensors, which detect when a person, in particular a hand of a person, approaches the handle 10 and an actuation of the handle 10 is imminent, or an actuation of the handle is performed.

In the exemplary embodiment according to FIG. 1, the disinfection container 50 is located within the rail vehicle door 20. Of course, it is possible with the exemplary embodiment according to FIG. 1 to fix the disinfectant container 50 on the outer side 30 of the rail vehicle door 20 or on the opposite side 31 of the rail vehicle door 20.

FIG. 2 shows the handle 10 according to FIG. 1 by way of example in a three-dimensional view from the side. It can be seen that the outer surface 90 of the handle 10 is provided with pores 80, through which the disinfectant of the disinfection device can exit.

FIG. 3 shows a second exemplary embodiment for a handle 10, which is connected to a disinfection device 40. In this exemplary embodiment too, the disinfection device 40 comprises a disinfectant container 50, in which a disinfectant 60 is located. The disinfectant container 50 is positioned spatially above the handle 10 in such a way that the disinfectant 60 can flow via a line 70 to the handle 10 as a result of the force of gravity.

In the exemplary embodiment according to FIG. 3, the outer surface 90 of the handle 10 is not provided with pores, but instead with a disinfectant-storing element 100, which consists entirely or at least in portions of an open-pore plastic, a rubber material or a sponge material and is capable of temporarily storing in a sponge-like manner disinfectant 60 delivered via the line 70. The disinfectant 60 then always exits from the disinfectant-storing element 100 when an operating person grasps the handle 10 and exerts a pressure onto the disinfectant-storing element 100.

In the exemplary embodiment according to FIG. 3, the disinfectant container 50 is located within the rail vehicle door 20, as is also the case in the exemplary embodiment according to FIGS. 1 and 2. Of course, it is also possible with the exemplary embodiment according to FIG. 3 to fix the disinfectant container 50 on the outer side 30 of the rail vehicle door 20 or on the opposite side 31 of the rail vehicle door 20.

FIG. 4 shows the handle 10 according to FIG. 3 with the disinfectant-storing element 100 by way of example in a three-dimensional view from the side. It can be seen that the disinfectant-storing element 100 is fixed to the outer surface 90 of the handle 10 and releases the disinfectant with exertion of a mechanical pressure.

FIG. 5 shows an exemplary embodiment for a handle 10, in which a disinfection device 40 is integrated. The disinfection device 40 comprises a pressure bottle 110, in which a disinfectant 60 is stored. The pressure bottle 110 is connected via a distributor 120 and a pressure limiter 130 to pores 80, which are fixed to the outer surface 90 of the handle 10. The pressure bottle 110 is at an overpressure, such that the disinfectant 60 located therein is pressed in the direction of the distributor 120 and pressure limiter 130. As a result of the overpressure in the pressure bottle 110, the disinfectant 60 is pressed in the direction of the pores 80 and can thus pass through the pores 80 onto the outer surface 90 of the handle 10. Here, the handle 10 or the outer surface 90 is thus disinfected by a continuous wetting of the outer surface 90 by the overpressure within the pressure bottle 110.

FIG. 6 shows a further exemplary embodiment for a handle 10, which is equipped with a disinfection device 40. The disinfection device 40 according to FIG. 6 is formed by a disinfectant-storing element 140 in the form of a jacket or sleeve. The disinfectant-storing element 140 is slid over the outer surface 90 of the handle 10 and thus covers this at least in portions.

The disinfectant-storing element 140 consists of a sponge-like material, which has the property of temporarily storing a disinfectant and dispensing this in small portions upon application of an external pressure. Thus, as soon as an operating person exerts pressure onto the disinfectant-storing element 140, disinfectant is dispensed through pores 142 of the element 140 and wets the outer surface 141 thereof, whereby both the outer surface 141 and the hand of the operating person contacting the handle 10 are disinfected.

FIG. 7 shows the handle 10 according to FIG. 6 by way of example in a three-dimensional view from the side. The disinfectant-storing element 140 can be seen, of which the outer surface 141 is provided with the pores 142, through which the disinfectant can pass onto the outer surface 141 as soon as pressure is exerted onto the disinfectant-storing element 140.

FIG. 8 shows a further exemplary embodiment for a handle 10, which is connected to a disinfection device 40. In the exemplary embodiment according to FIG. 8, the disinfection device 40 comprises a disinfectant container 50, in which the disinfectant 60 is housed. The disinfectant container 50 is connected via a line 70 to a spraying apparatus 200 in the form of a discharge nozzle, which sprays the disinfectant 60 onto the door handle 10.

In the exemplary embodiment according to FIG. 8, the disinfection device 40 and the door handle 10 are thus separated spatially from one another. A disinfection takes place by means of a misting or a spraying of the handle 10 by means of the distanced disinfection device 40 via the spraying apparatus 200. A triggering of the disinfection device 40 and a spraying or a misting of the door handle 10 is preferably triggered by means of a sensor 210, which detects an approach of a hand of an operating person and/or an actuation of the handle 10 and triggers a pump (not illustrated in FIG. 8 for reasons of clarity) of the disinfection device 40 before, after and/or during the actuation of the handle 10 in order to trigger a transport of the disinfectant 60 in the direction of the spraying apparatus 200 and therefore a spraying of the handle 10.

In the exemplary embodiment according to FIG. 8, the disinfectant container 50 is housed within the rail vehicle door 20. Alternatively, it is possible to fasten the disinfectant container 50 on the outer side 30 or on the opposite side 31 of the rail vehicle door 20.

FIG. 9 shows an exemplary embodiment for a handle 10, which is connected to a disinfection device 40 fastened on the outer side 30 of the rail vehicle door 20 and is disinfected by said disinfection device. The disinfection device 40 according to FIG. 9 is also equipped with a spraying apparatus 200, which disinfects the handle 10 by spraying or misting the door handle 10.

In order to enable a disinfection of the door handle 10 before, after and/or during the actuation of the handle 10, the disinfection device 40 is preferably equipped with a sensor 210, which detects a hand approaching the door handle 10 and/or an actuation of the door handle 10 and thus triggers, in accordance with a predefined control program, a transport of the disinfectant 60 in the direction of the spraying apparatus 200.

FIG. 10 shows an exemplary embodiment for a handle 10, of which the outer surface 90 is provided with pores, through which disinfectant can exit for the purpose of disinfecting the handle. The handle 10 according to FIG. 10, in terms of operation, corresponds to the handle 10 according to FIGS. 1 and 2, and merely differs in terms of its design. It can thus be seen that the handle 10 according to FIG. 10 is rectangular and not round.

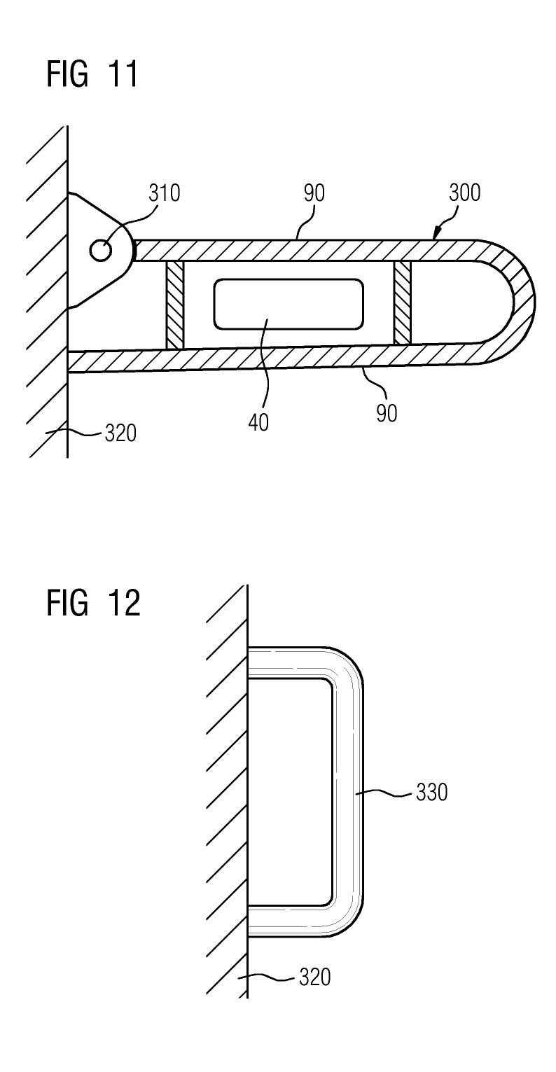

FIG. 11 shows an exemplary embodiment for a hinged handle 300, which is mounted pivotably about a bearing 310. The hinged handle 300 is preferably provided with a disinfection device 40, which is mounted within the hinged handle 300. By way of example, the outer surface 90 of the hinged handle 300 can be wetted via pores or disinfectant-storing elements, as has already been explained above in detail in conjunction with FIGS. 1 to 9.

FIG. 12 shows an exemplary embodiment for a grab handle 330, which is mounted fixedly on a support 320, for example a support wall. The grab handle 330 is also equipped with a disinfection device, which is not illustrated further in FIG. 12 for reasons of clarity. The disinfection device may correspond to one of the embodiments as already explained above in conjunction with FIGS. 1 to 9.

FIG. 13 shows an exemplary embodiment for a rail vehicle door 20, on the outer side 30 of which a first handle 10 is mounted and on the opposite side 31 of which a second handle 11 is mounted.

A disinfection device 40, which comprises a disinfectant container 50 containing disinfectant 60 is located within the rail vehicle door 20. The disinfectant container 50 is connected via lines 70 and 71 to the two handles 10 and 11 and supplies these with the disinfectant 60 for the purpose of disinfection.

In the exemplary embodiment according to FIG. 13 the disinfectant container 50 is located spatially below the two handles 10 and 11, such that either overpressure in the disinfectant container 50 or a pump is necessary for transport of the disinfectant 60. For reasons of clarity, such a pump is not illustrated in FIG. 13.

Alternatively, it is possible to arrange the disinfectant container 50 spatially above the two handles 10 and 11, such that transport of the disinfectant 60 to the two handles 10 and 11 is possible as a result of the force of gravity, as has been explained in detail already in conjunction with FIGS. 1 and 3.

A key advantage of the embodiment according to FIG. 13 lies in the fact that it is possible with a single disinfection device 40 and a single disinfectant container 50 to disinfect two handles 10 and 11. Within the scope of the maintenance of the disinfection device 40, it is therefore merely necessary to refill a single disinfectant container 50, whereby the maintenance outlay and therefore the maintenance costs are considerably reduced.

In addition, production costs are saved, since the number of components necessary for disinfection is lower than if each handle were equipped with an individual disinfection device.

Although the invention has been illustrated and described in greater detail by preferred exemplary embodiments, the invention is not limited by these disclosed examples, and other variations can be derived here from by a person skilled in the art without departing from the scope of protection of the invention.

1-12. (canceled)

13. A rail vehicle, comprising:
at least one handle for actuation by passengers or train staff;
said handle having a disinfection device or being connected to a disinfection device, said disinfection device being configured for disinfecting said handle before, during, and/or after an actuation of said handle.

14. The rail vehicle according to claim 13, wherein said at least one handle is a grab handle or an actuating handle.

15. The rail vehicle according to claim 14, wherein said handle is fixed in or to a wet room of the rail vehicle and said handle is configured to enable a person to hold steady within the wet room or to actuate a function of the wet room.

16. The rail vehicle according to claim 13, wherein said handle is a doorknob or a door handle of a rail vehicle toilet.

17. The rail vehicle according to claim 13, wherein said disinfection device comprises a disinfectant storage device containing a disinfectant.

18. The rail vehicle according to claim 17, wherein said disinfectant is a disinfecting liquid.

19. The rail vehicle according to claim 17, wherein said disinfectant storage device is selected from the group consisting of a sponge, an open-pore rubber element, and a pressure bottle.

20. The rail vehicle according to claim 17, wherein said disinfectant storage device is disposed in an interior of said handle or said disinfectant storage device is covered externally by an outer surface of said handle.

21. The rail vehicle according to claim 13, wherein said handle has an outer surface formed, at least in portions, with discharge openings enabling disinfectant to exit outwardly from an interior of said handle.

22. The rail vehicle according to claim 21, wherein said discharge openings are selected from the group consisting of pores, holes and nozzles.

23. The rail vehicle according to claim 17, wherein:
said handle is fixed to a fastening wall; and
said disinfectant storage device is affixed to a same side of, an opposite side of, or within said fastening wall.

24. The rail vehicle according to claim 23, wherein fastening wall is a door.

25. The rail vehicle according to claim 23, said disinfection device comprises at least one spraying apparatus disposed separately from said handle on said fastening wall, said spraying apparatus being configured to spray said handle with disinfectant after and/or during the actuation of the handle.

26. The rail vehicle according to claim 25, wherein said spraying apparatus is a spray nozzle.

27. The rail vehicle according to claim 13, wherein said disinfection device is a coating containing a disinfectant and said coating is applied completely or in portions to an outer surface of said handle.

28. The rail vehicle according to claim 13, wherein said at least one handle is one of a plurality of handles and said disinfection device is connected to the plurality of handles and configured for disinfecting said handles before, during, and/or after the actuation of the respective said handle.

29. A handle for a rail vehicle, comprising:
a disinfection device integrated with or connected to the handle, said disinfection device being configured for disinfecting the handle before, during, and/or after an actuation of the handle.

30. The handle according to claim 29, formed as a grab handle or an actuating handle for passengers or train staff in a rail vehicle.
